(19) United States
(12) Patent Application Publication
(10) Pub. No.: US 2020/0312194 A1
(43) Pub. Date: Oct. 1, 2020

(54) TRACHEOSTOMY SIMULATOR WITH FUNCTIONAL ANATOMICAL MODELS

(71) Applicant: Sandor Lunn Innovations, LLC, North Haven, CT (US)

(72) Inventors: Peter Sandor, North Haven, CT (US); James Lunn, North Haven, CT (US)

(73) Assignee: Sandor Lunn Innovations, LLC, North Haven, CT (US)

(21) Appl. No.: 16/821,413

(22) Filed: Mar. 17, 2020

Related U.S. Application Data
(60) Provisional application No. 62/827,335, filed on Apr. 1, 2019.

(51) Int. Cl. G09B 23/32; G09B 23/34; A61M 16/04

(52) U.S. Cl. CPC G09B 23/32; G09B 23/34; A61M 2205/80; A61M 2240/00; A61M 2210/1039; A61M 16/0488

(57) ABSTRACT

A simulated trachea for a tracheostomy simulator assembly includes a first conduit defining a trachea and a first aperture penetrating the sidewall of the trachea. The trachea is clear and surrounded by anatomically functional elements to provide efficient and thorough tracheostomy training for medical professionals.

### CROSS REFERENCE TO RELATED APPLICATION

This application claims the benefit of U.S. Provisional Patent Application Ser. No. 62/827,335 filed on Apr. 1, 2019, which is incorporated herein by reference in its entirety.

### FIELD OF THE INVENTION

The present invention is generally directed to a simulated trachea for a tracheostomy training device or simulator. More specifically, the present invention is directed to a novel assembly of a simulated trachea with other functional anatomical models to create a more realistic tracheostomy simulator assembly.

### BACKGROUND OF THE INVENTION

Tracheostomy simulators are an important tool for training medical staff. Currently available simulators are insufficient, because they do not show how different tracheostomy tubes sit in the trachea and how the tubes function. The interaction between different tracheostomy tubes and the lungs, the vocal cords, and the tracheostomy tube cuff is important. However, the currently available tracheostomy models teach the user to insert a tracheostomy tube into a blind hole in the trachea. Once in the tracheal hole, the user cannot see what is happening and how the device is functioning. This creates confusion, anxiety, and other complications.

Currently available tracheostomy simulators also do not have functional lungs or vocal cords. Functional anatomical models of both are necessary to cover all types of tracheostomy tube insertions to explain and show the interaction between the tracheostomy tube, the cuff and uncuffed type, the lungs, the vocal cords and an optional speaking valve.

There exists a need in the art for a tracheostomy simulator that is easy to use and provides more feedback for the user to allow the user to properly train for tracheostomy intubations.

### SUMMARY

There is disclosed herein a trachea simulator assembly for tracheostomy training that includes a conduit having an outer surface and an inner surface extending between an upstream end and a downstream end; a first aperture penetrating the conduit from the outer surface to the inner surface proximate to the upstream end; wherein the first aperture is configured to receive a tracheostomy tube therethrough and the conduit is substantially transparent; a voice simulation module upstream of the first aperture, the voice simulation module disposed in a flow passage defined by the inner surface, wherein the voice simulation module is configured to emit a sound when a gas passes therethrough; and an epiglottis simulation module positioned upstream of the voice simulation module and in fluid communication therewith, the epiglottis simulation module defined by a flap that is moveable between an open and closed position to regulate flow of the gas into the conduit.

In one embodiment, simulator assembly includes a valve located downstream of the first aperture and in fluid communication with the conduit, the valve configured to allow gas to flow in the downstream direction, out of the conduit. However, in preferred embodiments, no valve is employed.

In one embodiment wherein the valve is employed, the valve includes an actuator configured regulate gas to flow therethrough in the downstream direction and/or an upstream direction.

In one embodiment, simulator assembly includes one or more expandable vessels configured to simulate a human lung, the expandable vessel being in communication with the valve and positioned downstream thereof, the expandable vessel being configured to expand in volume in response to the flow of a gas therein and to contract in response to a release of gas therefrom.

In one embodiment, simulator assembly includes a first expandable vessel and a second expandable vessel configured to simulate human lungs, the first expandable vessel being in communication with a first valve and positioned downstream thereof, the second expandable vessel being in communication with a second valve and positioned downstream thereof, and the first and second expandable vessels being configured to expand in volume in response to the flow of a gas therein and to contract in response to a release of gas therefrom.

In one embodiment, simulator assembly includes a second conduit configured to simulate a human esophagus, the second conduit coupled to a mouth simulation module, the mouth simulation module is positioned upstream of the epiglottis simulation module and in fluid communication therewith.

In one embodiment, simulator assembly includes a stand releasably attached to a downstream end of the second conduit.

In one embodiment, simulator assembly includes a base fixed to the esophagus simulation module.

In one embodiment, all or part of the voice simulation module is removable.

In one embodiment, the valve resists the flow of gas in the upstream direction and/or the downstream direction.

In one embodiment, the conduit and first aperture accommodate a Pediatric tracheostomy and/or endotracheal tube.

In one embodiment, the conduit is centered on a longitudinal axis, and the conduit is transparent over 300 degrees relative to the longitudinal axis.

In one embodiment, two of the trachea assemblies, the voice simulation module, the epiglottis simulation model, the lung simulation module, and the esophagus simulation module interact to mimic a functioning human body.

### DESCRIPTION OF THE DRAWINGS

FIG. 1 is a side sectional view of a tracheostomy simulator assembly according to the present disclosure;

FIG. 2 is a side sectional view of the conduit of the tracheostomy simulator assembly of FIG. 1;

FIG. 3 is a side sectional view of the conduit of FIG. 2 with a tracheostomy tube and cuff depicted in phantom;

FIG. 4 is a photograph of a tracheostomy simulator assembly according to the present disclosure connected to a base;

FIG. 5 is a photograph of the tracheostomy simulator assembly of FIG. 4 with a manual inflation member attached;

FIG. 6 is a schematic perspective view of the tracheostomy simulator assembly of the present invention;

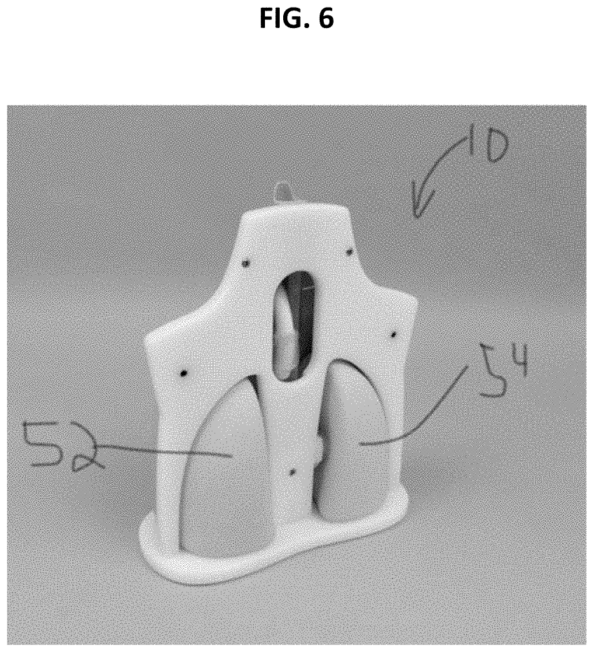

FIG. 7 is a schematic perspective view of the tracheostomy simulator assembly of the present invention shown with a cover in place;

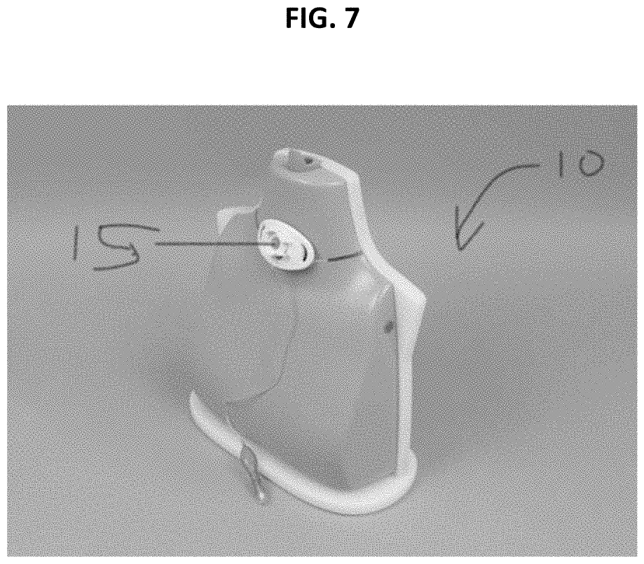

FIG. 8 is a schematic front view of the tracheostomy simulator assembly of the present invention shown with the cover off;

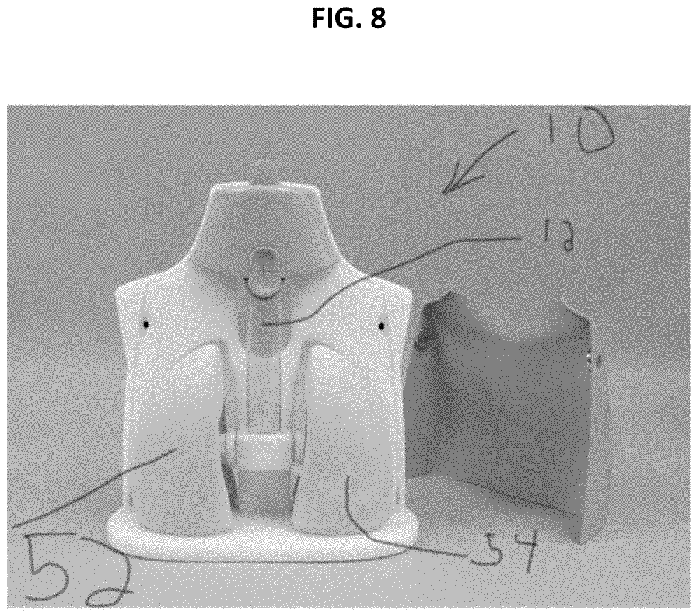

FIG. 9 is a schematic top view of the tracheostomy simulator assembly of the present invention; and

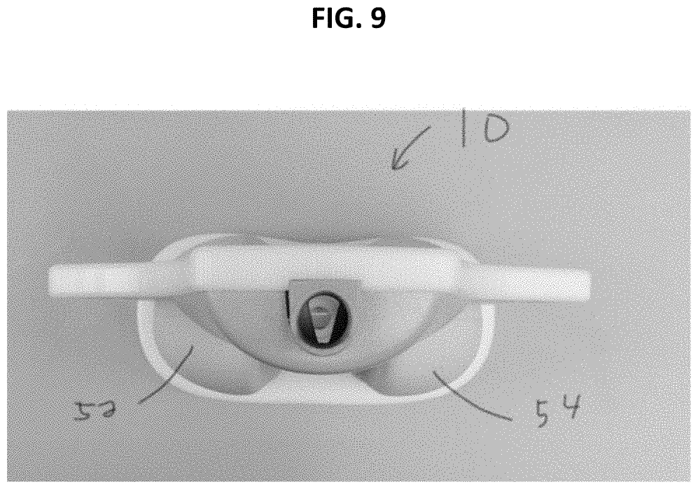

FIG. 10 is a schematic perspective view of the tracheostomy simulator assembly of the present invention.

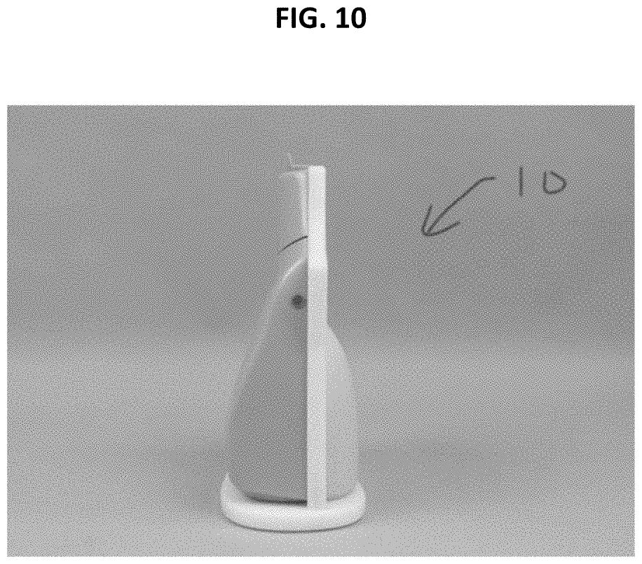

### DETAILED DESCRIPTION OF THE INVENTION

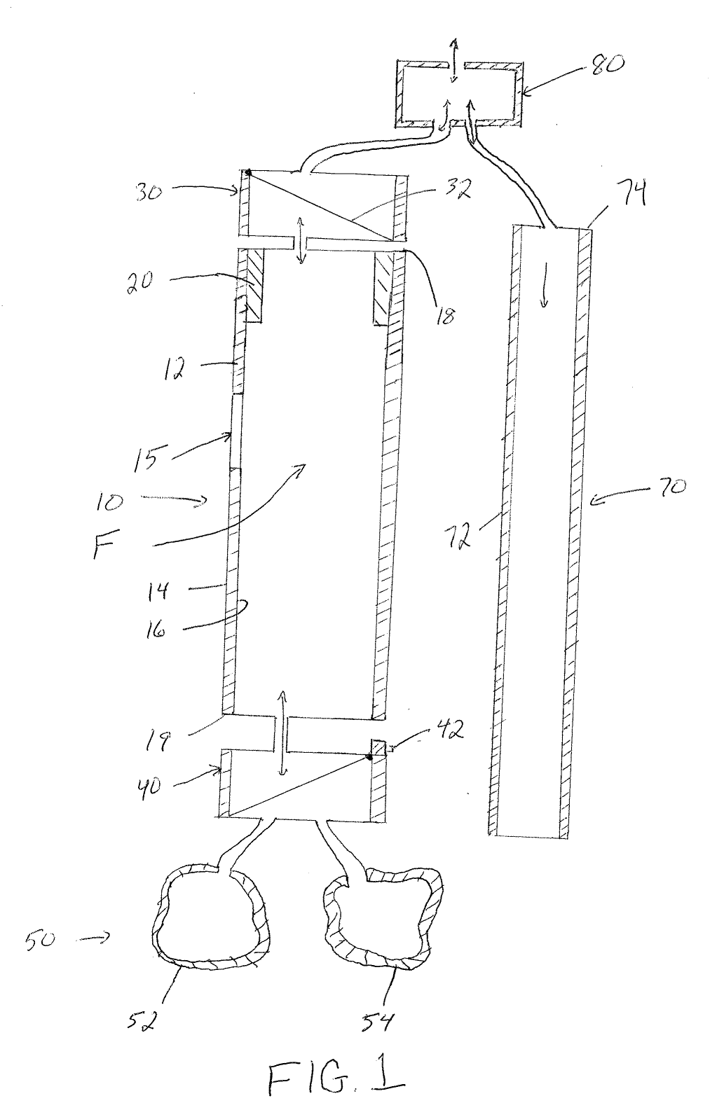

As shown in FIGS. 1-4, a trachea simulator assembly is generally designated by reference numeral 10. The trachea simulator assembly 10 is designed to improve tracheostomy training by providing anatomically correct elements and by improving the view of the trachea simulator assembly 10 before, during, and after intubation or tracheostomy tube insertion.

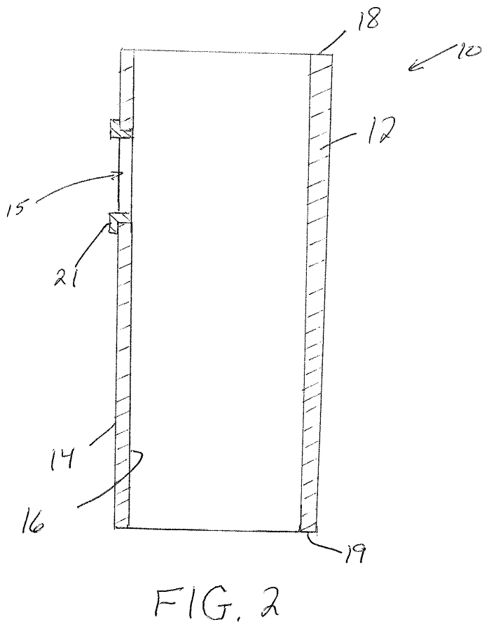

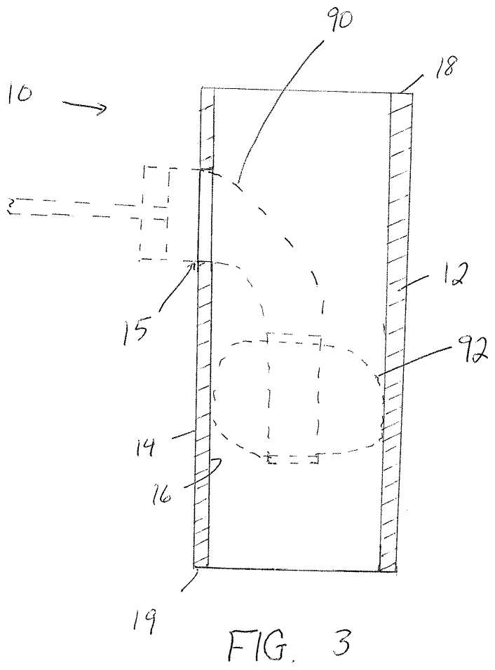

Referring to FIG. 2, the trachea simulator assembly 10 includes a conduit 12 extending between an upstream end 18 and a downstream end 19. The conduit 12 has an outer surface 14 and an inner surface 16. A first aperture 15 penetrates the conduit 12 from the outer surface 14 to the inner surface 16. In the depicted embodiment, the first aperture 15 is proximate to the upstream end 18 of the conduit 12. The first aperture 15 is designed to accommodate a tracheostomy tube including the inflating collar, as described below. In the depicted embodiment, a gasket 21 is retained in the first aperture 15. When a tracheostomy tube is inserted (as described below with reference to FIG. 3) it makes a seal around the tracheostomy tube.

Referring to FIG. 3, a tracheostomy tube 90 with collar 92 is depicted in phantom. The user inserts the first end of the tracheostomy tube 90 and collar 92 into the first aperture 15. The user then pushes the tracheostomy tube 90 a desired depth into the trachea simulator assembly 10. The user then inflates the collar 92, exerting a force against the inner surface 16 of the conduit 12. The trachea simulator assembly 10 is substantially transparent, allowing the user to view the tracheostomy tube 90 and collar 92 within the conduit 12 from a variety of angles to ensure proper depth and use of the collar 92. In some embodiments, the inside of the tracheostomy tube 90 is visible from between 300 and 360 degrees around the trachea simulator assembly 10. In other embodiments, a variety of tracheostomy tubes are compatible with the first aperture 15 of the conduit 12.

FIG. 1 depicts a voice simulation module 20, mounted within the upstream end 18 of the first conduit 12 of the trachea simulator assembly 10. The voice simulation module 20 is deposed in a flow passage defined by the inner surface 16 of the conduit 12. The voice simulation module 20 is configured to emit sound when a gas passes therethrough. In some embodiments, the voice simulation module 20 includes a reed retained by a ligature against a table. In some embodiments, the signal the voice simulation module 20 emits is a sound, a light, a vibration, or combinations thereof In some embodiments, the audible sound emitted by the voice simulation module 20 simulates human speech. In some embodiments, all or part of the voice simulation module 20 is removable to allow endotracheal (ETT) intubation.

In the embodiment depicted in FIG. 1, an epiglottis simulation module 30 is positioned upstream of the voice simulation module 20. The epiglottis simulation module 30 has a flap 32 that is moveable between an open position and a closed position. The flap 32 regulates the flow of a gas into the upstream end 18 of the conduit 12. In some embodiments, the flap 32 of the epiglottis simulation module 30 is in an anatomically correct position relative to the voice simulation module 20.

Referring to FIG. 1, in one embodiment, a valve 40 is attached to the downstream end 19 of the conduit 12. In the depicted embodiment, the valve 40 allows gases to flow in the downstream direction but prevents gas flow in the upstream direction. The valve 40 includes an actuator 42 that is configured to regulate gas flow through the valve 40. In the depicted embodiment, the valve 40 allows gas flow only in the downstream direction. When the actuator 42 is engaged the valve 40 also allows gas to flow in the upstream direction. In some embodiments, the valve 40 resists the flow of gas in the upstream and/or downstream direction. While the valve 40 is shown and described, in the preferred embodiment, no valve is employed. Two expandable vessels 52, 54 form the lung simulation module 50. The expandable vessels 52, 54 expand in volume in response to a flow of gas therein and contract in volume in response to a release of gas therefrom. In some embodiments, the expandable vessels 52, 54 automatically or self-inflate. In the depicted embodiment, the valve 40 has separate gas flow paths to each expandable vessel 52, 54 that are able to create resistance to gas flow. In some embodiments, each expandable vessel 52, 54 is connected to a separate valve.

FIG. 1 depicts a second conduit 72 that forms an esophagus simulation module 70. The second conduit 72 extends from an upstream first end 74 to a downstream second end 76.

The first end 74 of the second conduit 72 is in fluid communication with a mouth simulation module 80. The mouth simulation module 80 is upstream and in fluid communication with the esophagus simulation module 70 and the epiglottis simulation module 30.

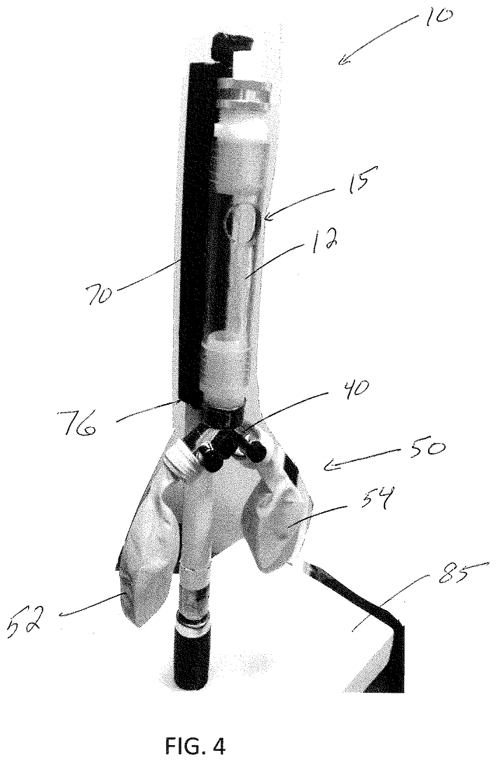

Referring to FIG. 4, the esophagus simulation module 70 connects to a base 85 at a downstream second end 76. In some embodiments, the base 85 is removable and/or the esophagus simulation module 70 is foldable relative to the base 85.

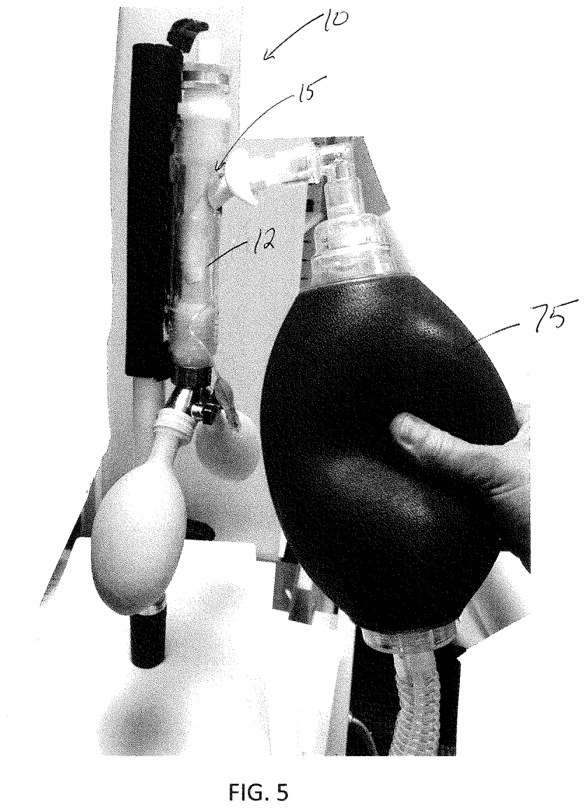

FIG. 5 depicts an gas delivery system 75 connected to the aperture 15 in the trachea simulator assembly 10. The depicted gas delivery system is manual, but other gas delivery systems, including automatic gas delivery systems do not depart significantly from the device disclosed herein.

In some embodiments, the conduit 12 and first aperture 15 accommodate a Pediatric tracheostomy tube and/or endotracheal tube.

In some embodiments, the tracheostomy simulator assembly is portable and lightweight.

In some embodiments, a soft tissue anterior or a hard plastic chest cover is attached to the trachea simulator assembly.

A trachea simulator assembly 10 for tracheostomy training includes a conduit 12 having an outer surface 14 and an inner surface 16 extending between an upstream end 18 and a downstream end 19; and a first aperture 15 penetrating the conduit 12 from the outer surface 14 to the inner surface 16 proximate to the upstream end 18, wherein the first aperture 15 is configured to receive a tracheostomy tube therethrough and the conduit 12 is substantially transparent.

A trachea simulator assembly 10 includes a voice simulation module 20 upstream of the first aperture 15, the voice simulation module 20 disposed in a flow passage defined by the inner surface 16, wherein the voice simulation module 20 is configured to emit a sound when a gas passes therethrough.

A trachea simulator assembly 10 includes an epiglottis simulation module 30 positioned upstream of the voice simulation module 20 and in fluid communication therewith, the epiglottis simulation module 30 defined by a flap 32 that is moveable between an open and closed position to regulate flow of the gas into the conduit 12.

A trachea simulator assembly 10 includes a valve 40 located downstream of the first aperture 15 and in fluid communication with the conduit 12, the valve 40 configured to allow gas to flow in the downstream direction, out of the conduit 12. In one embodiment, the valve 40 includes an actuator 42 configured regulate gas to flow therethrough in the downstream direction and/or an upstream direction.

A trachea simulator assembly 10 includes one or more expandable vessels 52, 54 configured to simulate a human lung 50, the expandable vessel 52, 54 being in communication with the valve 40 and positioned downstream thereof, the expandable vessel 52, 54 being configured to expand in volume in response to the flow of a gas therein and to contract in response to a release of gas therefrom.

A trachea simulator assembly 10 includes a first expandable vessel 52 and a second expandable vessel 54 configured to simulate human lungs 50, the first expandable vessel 52 being in communication with a first valve and positioned downstream thereof, the second expandable vessel 54 being in communication with a second valve and positioned downstream thereof, and the first and second expandable vessels 52, 54 being configured to expand in volume in response to the flow of a gas therein and to contract in response to a release of gas therefrom. While the first valve and the second valve are shown and described, in preferred embodiments no such valves are employed.

A trachea simulator assembly 10 includes a second conduit 72 configured to simulate a human esophagus 70, the second conduit 72 coupled to a mouth simulation module 80, the mouth simulation module 80 is positioned upstream of the epiglottis simulation module 30 and in fluid communication therewith.

A trachea simulator assembly 10 includes a stand releasably attached to a downstream end of the second conduit 72.

The present invention includes a voice simulation module 20 that includes a signal emitting member retained in a continuous flow path, wherein the voice simulation module 20 emits a signal (e.g., a sound, a vibration or a light) when gas passes therethrough. The signal emitting member includes a reed retained by a ligature against a table.

The present invention includes an epiglottis simulation module 30 that includes a flap 32 that is moveable between an open and closed position to regulate the flow of gas.

In certain embodiments wherein the valve 40 is employed, the present invention includes a lung simulation module 50, that includes a valve 40 configured to allow gas to flow in a downstream direction; one or more expandable vessels 52, 54 configured to simulate a human lung The expandable vessel 52, 54 is in communication with the valve 40 and positioned downstream thereof In one embodiment, the lung simulation module 50 includes a gas delivery system 75 connected to an aperture 15 in a trachea simulator assembly 10. In one embodiment, the gas delivery system 75 automatically delivers gas to the trachea simulator assembly 10.

The present invention includes an esophagus simulation module 70, that includes a second conduit 72 configured to simulate a human esophagus, the second conduit 72 coupled to a mouth simulation module 80 at an upstream end 74. In one embodiment, the mouth simulation module 80 is positioned upstream of an epiglottis simulation module 30 and in continuous communication therewith.

The present invention includes, a tracheostomy simulator assembly, that includes the trachea simulator assembly 10, the voice simulation module 20 mounted to an upstream end of the trachea simulator assembly 10; the epiglottis simulation module 30 mounted to an upstream end of the voice simulation module 20; the lung simulation module 50 mounted to a downstream end of the trachea simulator assembly 10; and the esophagus simulation module 70. In one embodiment, a base 85 is fixed to the esophagus simulation module 70. In one embodiment, all or part of the voice simulation module 20 is removable. In one embodiment wherein the valve is employed, the valve 40 resists the flow of gas in the upstream direction and/or the downstream direction. In one embodiment the conduit 12 and first aperture 15 accommodate a Pediatric tracheostomy and/or endotracheal tube. In one embodiment, the conduit is centered on a longitudinal axis, and the conduit is transparent over 300 degrees relative to the longitudinal axis. In one embodiment, the conduit is centered on a longitudinal axis, and the conduit is transparent about 360 degrees relative to the longitudinal axis.

Two of the trachea assemblies 10, the voice simulation module 20, the epiglottis simulation model 30, the lung simulation module 50, and the esophagus simulation module 70 interact to mimic a functioning human body.

While the present disclosure has been described with reference to various exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A trachea simulator assembly for tracheostomy training, the trachea simulator assembly comprising:
a conduit having an outer surface and an inner surface extending between an upstream end and a downstream end;
a first aperture penetrating the conduit from the outer surface to the inner surface proximate to the upstream end;
wherein the first aperture is configured to receive a tracheostomy tube therethrough and the conduit is substantially transparent;
a voice simulation module upstream of the first aperture, the voice simulation module disposed in a flow passage defined by the inner surface, wherein the voice simulation module is configured to emit a sound when a gas passes therethrough; and
an epiglottis simulation module positioned upstream of the voice simulation module and in fluid communication therewith, the epiglottis simulation module defined by a flap that is moveable between an open and closed position to regulate flow of the gas into the conduit.

2. The simulator assembly of claim 1, further comprising a valve located downstream of the first aperture and in fluid communication with the conduit, the valve configured to allow gas to flow in the downstream direction, out of the conduit.

3. The simulator assembly of claim 2, wherein the valve comprises an actuator configured regulate gas to flow therethrough in at least one of the downstream direction and an upstream direction.

4. The simulator assembly of claim 2, further comprising at least one expandable vessel configured to simulate a human lung, the expandable vessel being in communication with the valve and positioned downstream thereof, the expandable vessel being configured to expand in volume in response to the flow of a gas therein and to contract in response to a release of gas therefrom.

5. The simulator assembly of claim 2, further comprising a first expandable vessel and a second expandable vessel configured to simulate human lungs, the first expandable vessel being in communication with a first valve and positioned downstream thereof, the second expandable vessel being in communication with a second valve and positioned downstream thereof, and the first and second expandable vessels being configured to expand in volume in response to the flow of a gas therein and to contract in response to a release of gas therefrom.

6. The simulator assembly of claim 1, further comprising a second conduit configured to simulate a human esophagus, the second conduit coupled to a mouth simulation module, the mouth simulation module is positioned upstream of the epiglottis simulation module and in fluid communication therewith.

7. The simulator assembly of claim 6, further comprising a stand releasably attached to a downstream end of the second conduit.

8. The simulator assembly of claim 1, further comprising a base fixed to the esophagus simulation module.

9. The simulator assembly of claim 1, wherein all or part of the voice simulation module is removable.

10. The simulator assembly of claim 2, wherein the valve resists the flow of gas in the upstream direction and/or the downstream direction.

11. The simulator assembly of claim 1, wherein the conduit and first aperture accommodate a Pediatric tracheostomy and/or endotracheal tube.

12. The simulator assembly of claim 1, wherein the conduit is centered on a longitudinal axis, and the conduit is transparent over 300 degrees relative to the longitudinal axis.

13. The simulator assembly of claim 1, wherein at least two of the trachea assembly, the voice simulation module, the epiglottis simulation model, the lung simulation module, and the esophagus simulation module interact to mimic a functioning human body.
